(19) United States
(12) Patent Application Publication
(10) Pub. No.: US 2026/0279549 A1
(43) Pub. Date: Sep. 17, 2026

(54) BATCH DATA GENERATING METHOD, INFORMATION PROCESSING APPARATUS, LEARNING MODEL GENERATING METHOD, AND STORAGE MEDIUM

(71) Applicant: Konica Minolta, Inc., Tokyo (JP)

(72) Inventor: Takeshi YATSUZUKA, Tokyo (JP)

(21) Appl. No.: 19/560,326

(22) Filed: Mar. 9, 2026

(30) Foreign Application Priority Data
Mar. 17, 2025 (JP) 2025-042131

(51) Int. Cl. G16H 30/40

(52) U.S. Cl. CPC G16H 30/40

(57) ABSTRACT

A batch data generating method for generating batch data that is selected from population data including a plurality of medical images and that is used for learning by a learning model, the method including: classifying the population data into a plurality of categories; selecting an arbitrary category from the plurality of categories; setting an absolute number or a relative number of data to be selected from the population data in the batch data for each of the arbitrary category and the category other than the arbitrary category; and generating the batch data based on the absolute number or the relative number.

### CROSS-REFERENCE TO RELATED APPLICATION

The entire disclosure of Japanese Patent Application No. 2025-042131, filed on March 17, 2025, including description, claims, drawings and abstract is incorporated herein by reference.

### BACKGROUND OF THE INVENTION

### Technical Field

The present disclosure relates to a batch data generating method, an information processing apparatus, a learning model generating method, and a storage medium.

### DESCRIPTION OF RELATED ART

Conventionally, there is a diagnosis support apparatus that supports diagnosis by inferring a medical image using a learning model. In such a diagnosis support apparatus, inference accuracy of the medical image is important.

In Japanese Unexamined Patent Application Publication No. 2023-73825, object detection from an image using a learning model is described. An object of Japanese Unexamined Patent Application Publication No. 2023-73825 is to improve detection accuracy such as sensitivity and specificity.

Although Japanese Unexamined Patent Application Publication No. 2023-73825 is intended to uniformly improve the detection accuracy regardless of a category of the detection target, in actual clinical practice, the detection accuracy required by a user varies from category to category. For example, in the case of the diagnosis support apparatus, the detection accuracy for the category of severity (degree) is required to be higher than the detection accuracy for other categories. That is, a false negative that a human would not overlook leads to a user's distrust in the diagnostic support apparatus.

### SUMMARY OF THE INVENTION

An object of the present disclosure is to contribute to generation of a learning model in consideration of quality of detection accuracy in a particular category.

In order to solve the problem described above, according to an aspect of the present disclosure, a batch data generating method according to one aspect of the present disclosure is the batch data generating method for generating batch data that is selected from population data including a plurality of medical images and that is used for learning by a learning model, the method including:
classifying the population data into a plurality of categories;
selecting an arbitrary category from the plurality of categories;
setting an absolute number or a relative number of data to be selected from the population data in the batch data for each of the arbitrary category and the category other than the arbitrary category; and
generating the batch data based on the absolute number or the relative number.

According to another aspect, an information processing apparatus according to another aspect of the present disclosure is the information processing apparatus for generating batch data that is selected from population data including a plurality of medical images and that is used for learning by a learning model, the information processing apparatus including:
a hardware processor,
wherein the hardware processor executes,
classifying the population data into a plurality of categories,
selecting an arbitrary category from the plurality of categories,
setting an absolute number or a relative number of data to be selected from the population data in the batch data for each of the arbitrary category and the category other than the arbitrary category, and
generating the batch data based on the absolute number or the relative number.

According to another aspect, a storage medium according to another aspect of the present disclosure is the non-transitory computer-readable storage medium storing a program that is executed by a computer of an information processing apparatus for generating batch data that is selected from population data including a plurality of medical images and that is used for learning by a learning model, the program causing the computer to execute,
classifying the population data into a plurality of categories,
selecting an arbitrary category from the plurality of categories,
setting an absolute number or a relative number of data to be selected from the population data in the batch data for each of the arbitrary category and the category other than the arbitrary category, and
generating the batch data based on the absolute number or the relative number.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a block diagram illustrating a configuration of an information processing system;

FIG. 2 is a flowchart of a learning process;

FIG. 3 is an image diagram of batch creation;

FIG. 4 is the image diagram of the batch creation;

FIG. 5 is a flowchart of inference processing; and

FIG. 6 is the image diagram of the conventional batch creation.

### DETAILED DESCRIPTION

Hereinafter, one or more embodiments of the present disclosure will be described with reference to the drawings. However, the scope of the invention is not limited to the disclosed embodiments.

Hereinafter, embodiments of the present disclosure will be described with reference to the drawings. However, the scope of the present disclosure is not limited to the following embodiment and drawings.

### Information Processing System 100

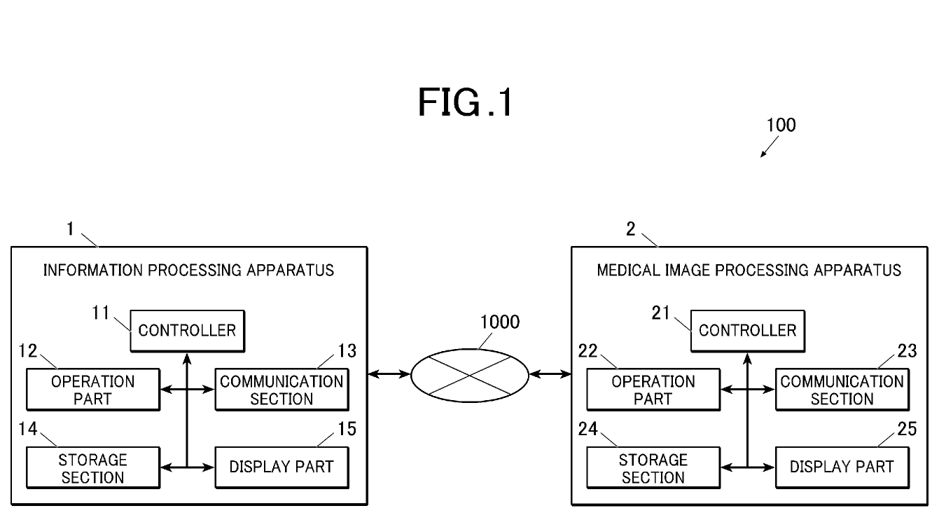

First, a configuration of an information processing system 100 will be described with reference to FIG. 1. As illustrated in FIG. 1, the information processing system 100 is configured by connecting an information processing apparatus 1 and a medical image processing apparatus 2 via a communication network 1000 such as a local area network (LAN).

The information processing apparatus 1 is an apparatus for a user who provides a learning model to cause the learning model to perform learning by using batch data selected from population data including a plurality of medical images.

The medical image processing apparatus 2 is an apparatus for the user such as a doctor to cause a provided learning model to execute inference such as image analysis of the medical image.

### Information Processing Apparatus 1

Next, a configuration of the information processing apparatus 1 will be described with reference to FIG. 1. As illustrated in FIG. 1, the information processing apparatus 1 includes a controller 11, an operation part 12, a communication section 13, a storage section 14, and a display part 15.

The controller 11 includes a central processing unit (CPU), a random access memory (RAM), and the like. The CPU of the controller 11 reads various programs stored in the storage section 14, deploys the programs in the RAM, executes various processes in accordance with the deployed programs, and controls the operation of each section of the information processing apparatus 1.

The controller 11 functions as a classification section that classifies the population data into a plurality of categories for each item.

The item is the item for categorizing the medical image, and is based on, for example, a clinical case.

Specifically, the items and the categories include the following examples.

### Specific Example of Item and Category

Item: Category

Case name: case A/case B/case C, etc.

Severity of case: severe/moderate/mild/ultra-mild, etc.

Finding positions of cases: upper lung/lower lung/left lung/right lung/lung periphery/vicinity of mediastinum, etc.

Case findings clarity: clear/unclear, etc.,

Alternatively, the item may be the detection difficulty level of the case in which the severity, the finding position, and the clarity of the case are complexly considered.

### Specific Example of Item and Category

Item: Category

Detection difficulty: difficult/normal/easy, etc.

Further, a plurality of different items may be hierarchized. Specific examples will be described later.

The controller 11 functions as a selection section that selects an arbitrary category from a plurality of categories.

The arbitrary category to be selected is not limited to one, and may be two or more.

The controller 11 functions as a setting section that sets a selection ratio for each of an arbitrary category and the category other than the arbitrary category in the batch data. The selection ratio is a relative number such as a percentage with respect to the entire population data or a ratio between the arbitrary category and another category. The selection ratio may be an absolute number such as a number of data or an amount of data selected from the population data.

The selection ratio is a ratio of data of the arbitrary category and the category other than the arbitrary category in the batch data selected from the population data. Specific examples will be described later.

Note that the selection ratio may be set on the basis of a learning stance selected by the user. The learning stance is a policy for causing the learning model to perform learning, and is, for example, a scene in which the learning model is used or a target user who uses the learning model. A specific example of the setting will be described later.

The controller 11 functions as a generation section that generates the batch data based on the selection ratio.

The controller 11 also functions as a learning model construction section to generate a learning model using the batch data.

The operation part 12 includes a keyboard having cursor keys, number input keys, and various function keys, a pointing device such as a mouse, and a touch screen layered on a surface of the display part 15. The operation part 12 is operable by an operator. The operation part 12 outputs various signals based on an operation performed by the operator to the controller 11.

The communication section 13 can transmit and receive various signals and various data to and from other devices and the like that are connected for communication via the network 1000.

The storage section 14 includes a nonvolatile semiconductor memory a hard disk, and the like, and stores various programs to be executed by the controller 11, parameters necessary for executing the programs, various data, and the like.

The storage section 14 stores the learning model, the population data, and the like.

The display part 15 includes a monitor such as a liquid crystal display (LCD) and displays various screens and the like in accordance with an instruction of a display signal input from the controller 11.

### Medical Image Processing Apparatus 2

Next, a configuration of the medical image processing apparatus 2 is described with reference to FIG. 1. As illustrated in FIG. 1, the medical image processing apparatus 2 includes a controller 21, an operation part 22, a communication section 23, a storage section 24, and a display part 25.

The controller 21 includes a CPU, a RAM, and the like. The CPU of the controller 21 reads various programs stored in the storage section 24, deploys the programs in the RAM, executes various processes in accordance with the deployed programs, and controls the operation of each section of the medical image processing apparatus 2.

The controller 21 functions as an inference execution section that executes medical image inference by using the learning model learned by using the batch data selected from the population data including a plurality of medical images.

The controller 21 functions as a selection section that selects the learning model to be caused to perform inference. Note that the storage section 24 described later may store a plurality of learning models learned by using batch data with different selection ratios.

The operation part 22 includes a keyboard having cursor keys, number input keys, and various function keys, a pointing device such as a mouse, and a touch screen layered on the surface of the display part 25. The operation part 22 is operable by an operator. The operation part 22 outputs various signals based on an operation performed by the operator to the controller 21.

The communication section 23 can transmit and receive various signals and various data to and from other devices and the like that are connected for communication via the network 1000.

The storage section 24 includes a nonvolatile semiconductor memory, a hard disk, and the like, and stores various programs to be executed by the controller 21, parameters necessary for executing the programs, various data and the like.

The storage section 24 stores the learning model.

The display part 25 includes a monitor such as a liquid crystal display (LCD), and displays various screens and the like in accordance with the instruction of the display signal input from the controller 21.

### Learning Process

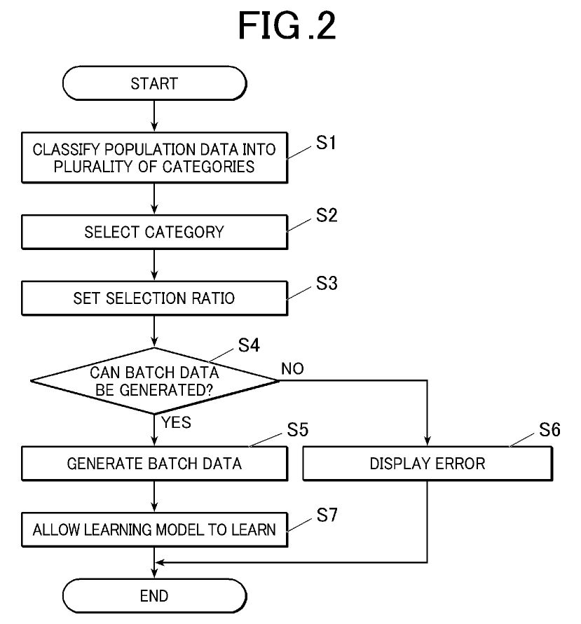

Next, a learning process of causing the learning model to perform learning in the information processing apparatus 1 will be described with reference to FIG. 2.

First, the user selects an item using the operation part 12. The controller 11 classifies the population data into a plurality of categories (step S1; classifying step).

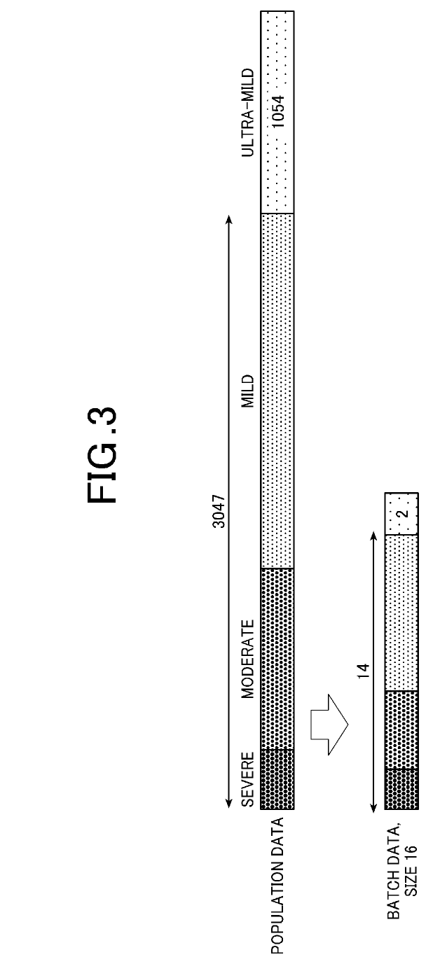

Here, an image of classifying population data into a plurality of categories will be described with reference to FIG. 3.

FIG. 3 is an image diagram of batch data generation. The upper row is a bar graph in which the population data (severe/moderate/mild; 3047 examples, ultra-mild; 1054 examples) is classified into a plurality of categories. In the example of FIG. 3, the item is an example of one item of the severity of the case, and the population data is classified based on a plurality of categories of severe/moderate/mild/ultra-mild.

Next, the user selects the arbitrary category using the operation part 12. The controller 11 selects the arbitrary category (step S2; selection step).

The example of FIG. 3 is an example in which the category of ultra-mild is selected.

Next, the user sets the selection ratio by using the operation part 12. The controller 11 sets the selection ratio (step S3; setting step).

The example of FIG. 3 is an example in which the size of the batch data is 16 and the selection ratio is set to the selection ratio of 7:1 for severe/moderate/mild/ultra-mild.

Next, the controller 11 determines whether the batch data generation can be performed at the selection ratio (step S4). When it is determined that the batch can be generated (step S4; YES), the controller 11 advances the learning process to step S5. When it is determined that the batch cannot be generated (step S4; NO), the controller 11 advances the learning process to step S6.

The controller 11 makes the determination based on the size of the batch data and the selection ratio. For example, in the example of FIG. 3, assuming that the number of data on severe/moderate/mild in the population data is 13 or less, the controller 11 determines that it is not possible to generate the batch data having the size of 16 and the selection ratio of 7:1 (severe/moderate/mild/ultra-mild).

Next, the controller 11 generates the batch data based on the selection ratio (step S5; generating step).

In the example of FIG. 3, the batch data is generated by randomly extracting 14 cases from the data of severe/moderate/mild of the population data and 2 cases from the data of ultra-mild of the population data. In this case, about half of the ultra-mild data of the population data is used for learning in one epoch.

Next, the controller 11 displays an error message on the display part 15 (step S6).

For example, the controller 11 displays on the display part 15 an error message such as “Batch data cannot be generated with such size and selection ratio". Note that the controller 11 may cause the display part 15 to display, for example, the size and the selection ratio with which the batch data can be generated.

Next, the controller 11 causes the learning model to learn using the created batch data (step S7; learning model generation step), and ends the learning process.

In the above description, the case where the item selected in step S1 is one item of the severity of the case has been described, but the present disclosure is not limited to this example. A plurality of items may be included.

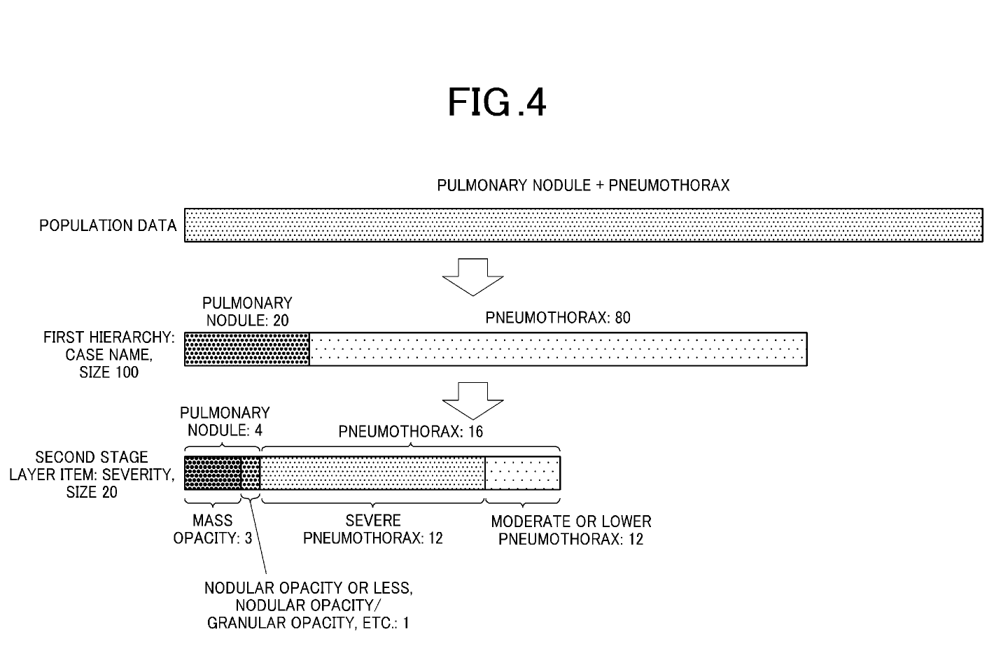

The batch data generation when a plurality of (two) items are selected will be described with reference to FIG. 4.

FIG. 4 is an example in which the population data includes a pulmonary nodule and pneumothorax. The item in the first hierarchy is a case name, the size of the batch data is 100, and the selection ratio is 1:4 (pulmonary nodule: pneumothorax), and the cases are randomly extracted. Next, the item at the second hierarchy is severity, the batch data size is 20, the selection ratio is 3:1 (mass opacity (severe): nodule opacity or less) and 3:1 (severe pneumothorax (severe): moderate or less pneumothorax), and they are extracted at random. Note that although the selection ratio of severity is applied to both the pulmonary nodule and the pneumothorax in the second hierarchy, it may be applied to only one of them.

In a case where the number of items is three or more, similarly, the population data may be hierarchically classified.

In this way, the learning model is generated in consideration of quality of detection accuracy in a particular category.

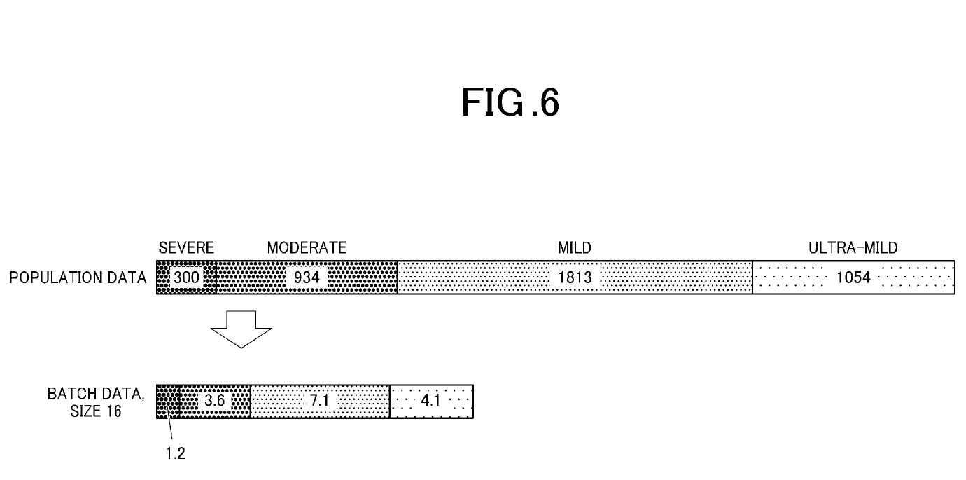

Note that FIG. 6 is an example of conventional batch creation. In this case, since the ratio of the number of data in each category in the population data directly becomes the selection ratio of the batch data, learning may be excessively pulled by the ultra-mild data more than the user feels. In this case, all data of the ultra-mild data of the population data is used for learning in one epoch.

### Inference Processing

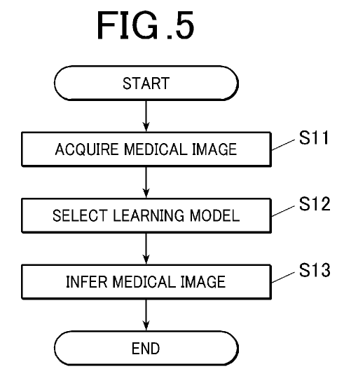

Next, a medical image inference process in the medical image processing apparatus 2 will be described with reference to FIG. 5.

First, the user selects, using the operation part 22, the medical image that is the target for which the learning model is caused to perform inference. The controller 21 acquires the medical image (step S11).

Next, the user selects the learning model to be used, using the operation part 22. The controller 21 selects the learning model (step S12; model selection step).

For example, the storage section 24 may store a first learning model learned using the batch data having the size of 16 and the selection ratio of 1:7 (severe/moderate : mild/ultra-mild, item; severity of case) and a second learning model learned using the batch data having the size of 16 and the selection ratio of 7:1 (severe : moderate/mild/ultra-mild, item; severity of case). In such a case, the user can switch between the first learning model and the second learning model, and thus can cope with a desired case.

Next, the controller 21 infers the medical image using the learning model (step S13; inference execution step), and ends the inference processing.

In this way, since quality of accuracy is considered in the learning model, the user can obtain an inference result that is along his/her sense.

### Application Example

The above is applicable to the following cases.

Here, an application example will be described for each learning stance (scene, target user, and oversight prevention).

(Scene) • Medical examination site: Since it is assumed that there are many healthy persons with hardly any pneumothorax findings, the selection ratio is set such that the learning ratio when the severity of the case is mild or less is higher than the learning ratio when the severity of the case is moderate or more, thereby improving sensitivity to these cases.

Emergency or inpatient: Since it is assumed that lying position imaging is often performed in a situation such as in a bed, the selection ratio is set such that the learning ratio of lower lung field findings is high to improve sensitivity to these findings.

Emergency transportation or emergency outpatient due to a traffic accident or sudden dyspnea: Since there is a high possibility that a moderate or higher pneumothorax finding is recognized, the selection ratio is set such that the moderate or severe learning ratio is high to improve the sensitivity to them.

### Target User

In a case where there is a difference in image reading skill between a doctor and a technologist or between a radiologist and a non-radiologist, the selection ratio is set to be high for findings that the skill is poor to improve sensitivity to such findings.

### Prevention of Oversight

Generally, since a finding of mild or less is easily overlooked due to the smallness of the finding, the selection ratio is set such that the learning ratio of mild or less is high.

When the findings are superimposed on the ribs or the multiple organs, it is difficult to recognize the finding features, and thus the selection ratio is set such that the learning ratio of the findings located on these positions is high.

The follow-up observation is possible when mild or less, but it is not desired to be missed at all when moderate or more because there is a risk of severity later on and a risk of litigation due to the risk of severity. Therefore, the selection ratio is set such that the learning ratio of moderate or more is high.

In the case of detecting both the nodule/infiltrative shadow and pneumothorax, the selection ratio is set such that the learning ratio of pneumothorax is high because it is desired to preferentially detect pneumothorax from the viewpoint of urgency.

When the slice thickness is small, it is required to detect a finding having a small size, and thus the selection ratio is set such that the learning ratio of the finding having a small size is higher than that when the slice thickness is large.

Note that although it has been described above that the selection ratio may be set on the basis of the learning stance, for example, the selection ratio may be stored in the storage section 14 of the information processing apparatus 1 in association with each of the above-described application examples, that is, each learning stance, and the user may select the learning stance to set the selection ratio. In this way, the user can easily set the selection ratio.

### Others

In the above description, the learning model and the population data are stored in the storage section 14 of the information processing apparatus 1 and the learning model is stored in the storage section 24 of the medical image processing apparatus 2, but they may be stored in an external server (an on-premise server, a cloud server, or the like).

Further, when two or more arbitrary categories are selected, the plurality of arbitrary categories may be collectively handled as one arbitrary category. To be specific, when the item is “severity of case" and the selected arbitrary category is "severe" or "moderate", the selection ratio may be A:B (severe/moderate: mild/ultra-mild) or A:B:C (severe: moderate: mild/ultra-mild).

### Effect

As described above, the batch data generating method (learning process) is the batch data generating method of generating the batch data selected from the population data including the plurality of medical images and used for learning by the learning model, and includes the classifying step (step S1) of classifying the population data into the plurality of categories, the selecting step (step S2) of selecting the arbitrary category from the plurality of categories, the setting step (step S3) of setting the absolute number or a relative number of data selected from population data in the batch data for each of the arbitrary category and the category other than the arbitrary category, and the generating step (step S5) of generating the batch data based on the absolute number or the relative number.

Therefore, it is possible to contribute to the generation of the learning model in consideration of the quality of detection accuracy in a particular category.

The information processing apparatus 1 is the information processing apparatus for generating the batch data selected from the population data including the plurality of medical images and used for learning by the learning model, and includes, the classification section (controller 11) for classifying the population data into the plurality of categories; the selection section (controller 11) for selecting the arbitrary category from the plurality of categories; the setting section (controller 11) for setting the absolute number or the relative number of the data selected from the population data in the batch data of each of the arbitrary category and the category other than the arbitrary category; and the generation section (controller 11) for generating the batch data on the basis of the absolute number or the relative number.

Therefore, it is possible to contribute to the generation of the learning model in consideration of the quality of detection accuracy in a particular category.

The program causes the computer of the information processing apparatus 1 that generates the batch data selected from the population data including the plurality of medical images and used for learning by the learning model to function as the classification section (controller 11) that classifies the population data into the plurality of categories, the selection section (controller 11) that selects the arbitrary category from the plurality of categories, the setting section (controller 11) that sets the absolute number or the relative number of the data selected from the population data in the batch data for each of the arbitrary category and categories other than the arbitrary category, and the generation section (controller 11) that generates the batch data based on the absolute number or the relative number.

Therefore, it is possible to contribute to the generation of the learning model in consideration of the quality of detection accuracy in a particular category.

The medical image processing apparatus 2 includes the inference execution section (controller 21) that executes the inference of the medical image using the learning model learned using the batch data selected from the population data including the plurality of medical images, and the batch data is the batch data selected from the population data such that the absolute number or the relative number of the data selected from the population data in the batch data for each of the arbitrary category in categorized items and the category other than the arbitrary category is the set absolute number or relative number.

Therefore, the inference can be performed by using the learning model in which the quality of the detection accuracy in the specific category is considered.

Hereinabove, the present disclosure has been specifically described with reference to the embodiment, but the present disclosure is not limited to the above-described embodiment and can be appropriately modified without departing from the scope of the present disclosure.

For example, although a hard disk, a semiconductor nonvolatile memory, or the like is used in the above description as a computer-readable medium storing the program according to the present disclosure, the present disclosure is not limited to this example. As other computer-readable storage media, portable storage media such as CD-ROMs can be applied.

The detailed configuration and the detailed operation of each component can be appropriately changed without departing from the scope of the invention.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. A batch data generating method for generating batch data that is selected from population data including a plurality of medical images and that is used for learning by a learning model, the method comprising:
classifying the population data into a plurality of categories;
selecting an arbitrary category from the plurality of categories;
setting an absolute number or a relative number of data to be selected from the population data in the batch data for each of the arbitrary category and the category other than the arbitrary category; and
generating the batch data based on the absolute number or the relative number.

2. The batch data generating method according to claim 1, wherein,
in the classifying, the population data is classified into the plurality of categories for each item, and
the item is based on a clinical case.

3. The batch data generating method according to claim 1, wherein,
in the classifying, the population data is classified into the plurality of categories for each item, and
the item is hierarchized.

4. The batch data generating method according to claim 1, wherein, in the setting, the absolute number or the relative number is set based on a learning stance.

5. An information processing apparatus for generating batch data that is selected from population data including a plurality of medical images and that is used for learning by a learning model, the information processing apparatus comprising:
a hardware processor,
wherein the hardware processor executes,
classifying the population data into a plurality of categories,
selecting an arbitrary category from the plurality of categories,
setting an absolute number or a relative number of data to be selected from the population data in the batch data for each of the arbitrary category and the category other than the arbitrary category, and
generating the batch data based on the absolute number or the relative number.

6. A non-transitory computer-readable storage medium storing a program that is executed by a computer of an information processing apparatus for generating batch data that is selected from population data including a plurality of medical images and that is used for learning by a learning model, the program causing the computer to execute,
classifying the population data into a plurality of categories,
selecting an arbitrary category from the plurality of categories,
setting an absolute number or a relative number of data to be selected from the population data in the batch data for each of the arbitrary category and the category other than the arbitrary category, and
generating the batch data based on the absolute number or the relative number.

7. A learning model generating method comprising:
generating a learning model using the batch data according to claim 1.

8. An information processing apparatus comprising:
a hardware processor that generates a learning model using the batch data according to claim 5.

9. A non-transitory computer-readable storage medium storing a program that causes a computer of an information processing apparatus that generates a learning model to execute,
training a learning model using the batch data of claim 6.
